(19) United States
(12) Patent Application Publication
(10) Pub. No.: US 2012/0041065 A1
(43) Pub. Date: Feb. 16, 2012

(54) LAURIC ACID DISTILLATE FOR ANIMAL FEED

(75) Inventors: William Guy Coxon Appleby, Newbury (GB); David A. Cook, Coon Rapids, MN (US); Kim Gene Friesen, Carthage, IN (US); David A. Pascoe, Elk River, MN (US); Michael C. Thiede, Maple Grove, MN (US)

(73) Assignees: CAN TECHNOLOGIES, INC., Hopkins, MN (US); ELI LILLY AND COMPANY, Indianapolis, IN (US)

(21) Appl. No.: 13/193,654

(22) Filed: Jul. 29, 2011

Related U.S. Application Data
(60) Provisional application No. 61/372,261, filed on Aug. 10, 2010.

(51) Int. Cl. A61K 31/20

(52) U.S. Cl. 514/558

(57) ABSTRACT

Provided are novel methods and formulations for enhancing feed efficiency and reducing mortality in food animals.

Enhancing animal growth or feed efficiency can have substantial impact on, for example, the animal meat industry by reducing the high cost of feeding and maintaining food-producing animals, thus directly improving profitability. For example, in the poultry industry, even a slight increase in broiler growth rate coupled with reduced feed consumption brings the broiler to market maturity faster at a lower cost. With more than eight billion broilers raised annually just in the United States, significant savings are realized for even small or incremental enhancements in the animal's growth and/or efficiency. Further, reduced mortality in food animals positively impacts the profitability of producing food animals.

Lauric acid is a naturally occurring twelve carbon fatty acid found in plant oils, such as palm kernel and coconut. In purifying the plant oils, the crude oil concentrates are subject to a distillation process which results in a distillate having a concentration of lauric acid normally in the range of 45-55% of lauric acid, along with lesser amounts of glycerol and other fatty acids. This lauric acid distillate is generally considered a waste product, particularly in Malaysia, the world's largest producer of palm kernel oil, and is burned as fuel or used in soap manufacture. The palm kernel fatty acid distillate is sold as fuel oil in other parts of the world.

The present invention encompasses methods and formulations for enhancing growth and/or feed efficiency in animals, and particularly food animals, using enhanced lauric acid distillate such as hydrolyzed lauric acid distillate. The invention further encompasses methods and formulations for reducing mortality in animals, and particularly food animals, using enhanced lauric acid distillate, such as hydrolyzed lauric acid distillate. Additionally, methods and formulations are provided for enhancing breast and leg meat yield in poultry using enhanced lauric acid distillate, such as hydrolyzed lauric acid distillate. Also provided are enhanced lauric acid distillate compositions.

Animals include, but are not limited to, farm livestock including equine animals, companion animals (e.g. pets such as dogs and cats), and ruminant and monogastric food animals whose meat is used, or who produce items, for human consumption. Poultry, such as chickens, turkeys, ducks, pheasant and quail, fish, shrimp, porcine animals (e.g. pigs), ovine animals (e.g. lambs and sheep), and bovine animals (e.g. cattle, including dairy cattle), are examples of food animals.

Lauric acid distillate is the byproduct which results from the distillation process to obtain purified plant oils, such as palm kernel and coconut. Enhanced lauric acid distillate is lauric acid distillate which has been further modified, such as by increasing the overall lauric acid content to up to 75%, by being hydrolyzed, and/or being further distilled. Hydrolyzed lauric acid distillate is lauric acid distillate which has undergone hydrolysis in order to increase the conversion of the mono-, di-, and triglycerides in the distillate to glycerol and free fatty acids. The lauric acid in the distillate is de-esterified from the glycerol backbone which enhances its characteristics. The hydrolysis conversion is preferably substantially 100%, but hydrolyzed lauric acid distillate includes conversions less than 100%, though a substantial conversion is desired, that being at least 40%. The hydrolyzed lauric acid distillate is normally between 45-55% lauric acid, and includes other materials such as myristic, palmitic, stearic, oleic, caproic, caprylic, capric, and linoleic acids, though these other materials are present in a lower amount than lauric acid, normally each less than 20%. When distillation is employed to enhance the distillate, the amounts of some or all of the fatty acids may be reduced, such as a reduction of palmitic acid to low levels.

Examples of the components in a lauric acid distillate or enhanced lauric acid distillate, which may be blended from more than one distillation process, can be, by weight percent:

| | |
|---|---|
| Caproic acid | 0.1-0.7 |
| Caprylic acid | 4.6-6.3 |
| Capric acid | 4.3-5.2 |
| Lauric acid | 45-75 |
| Myristic acid | 15.1-16.6 |
| Palmitic acid | 7.4-9.0 |
| Stearic acid | 2.1-2.8 |
| Oleic acid | 8.4-10.5 |
| Linoleic acid | 1.3-1.9 |

More specific hydrolyzed palm kernel oil distillate compositions 1-6, in percentages, are as follows in Table 1a:

TABLE 1a

| COMPONENT | 1 | 2 | 3 | 4 | 5 | 6 |
|---|---|---|---|---|---|---|
| C06:0 Caproic Acid - 6:0 (% of FA) | 0.17 | 0.12 | 0.24 | 0.26 | 0.18 | 0.194 |
| C08:0 Caprylic Acid - 8:0 (% of FA) | 4.69 | 4.56 | 5.49 | 4.76 | 4.64 | 4.828 |
| C10:0 Capric Acid - 10:0 (% of FA) | 4.53 | 4.36 | 5.1 | 4.52 | 4.42 | 4.586 |
| C12:0 Lauric Acid - 12:0 (% of FA) | 52.23 | 52.45 | 51.86 | 52.28 | 53.61 | 52.49 |
| C14:0 Myristic Acid - 14:0 (% of FA) | 16.59 | 16.37 | 15.11 | 16.1 | 16.17 | 16.07 |
| C16:0 Palmitic Acid - 16:0 (% of FA) | 8.99 | 8.95 | 7.48 | 8.55 | 8.53 | 8.5 |
| C18:0 Stearic Acid - 18:0 (% of FA) | 2.78 | 2.57 | 2.56 | 2.58 | 2.45 | 2.588 |
| C18:1 9c Oleic Acid - 18:1 Oleic (% of FA) | 8.41 | 9.02 | 10.41 | 9.2 | 8.46 | 9.1 |
| C18:2 9c12c Linoleic Acid - 18:2 Linoleic (% of FA) | 1.55 | 1.49 | 1.71 | 1.51 | 1.37 | 1.526 |

Compositions 7 and 8 are provided as follows in Tables 1b and 1c, respectively.

TABLE 1b

| COMPONENT | 7 |
|---|---|
| C06:0 Caproic Acid - 6:0 | 0.61 |
| C08:0 Caprylic Acid - 8:0 | 6.28 |
| C10:0 Capric Acid - 10:0 | 5.05 |
| C11:0 Hendecanoic Acid - 11:0 | 0.2 |
| C12:0 Lauric Acid - 12:0 | 49.74 |
| C14:0 Myristic Acid - 14:0 | 15.34 |
| C16:0 Palmitic Acid - 16:0 | 8.23 |
| C18:0 Stearic Acid - 18:0 | 2.19 |
| C18:1 9c Oleic Acid - 18:1 Oleic | 9.12 |
| C18:2 9c12c Linoleic Acid - 18:2 Linoleic | 1.87 |
| C20:0 Arachidic Acid - 20:0 | 0.17 |
| C20:3 Homo-y Linolenic Acid - 20:3 | 0.18 |

TABLE 1c

| COMPONENT | 8 |
|---|---|
| C06:0 Caproic Acid - 6:0 | — |
| C08:0 Caprylic Acid - 8:0 | 7.34 |
| C10:0 Capric Acid - 10:0 | 7.96 |
| C11.0 Hendecanoic Acid - 11:0 | 0.67 |
| C12:0 Lauric Acid - 12:0 | 56.75 |
| C14:0 Myristic Acid - 14:0 | 18.23 |
| C16:0 Palmitic Acid - 16:0 | 5.89 |
| C18:0 Stearic Acid - 18:0 | 0.96 |
| C18:1 9c Oleic Acid - 18:1 Oleic | 1.65 |
| C18:2 9c12c Linoleic Acid - 18:2 Linoleic | 0.5 |
| C20:0 Arachidic Acid - 20:0 | — |
| C20:3 Homo-y Linolenic Acid - 20:3 | — |

Feed efficiency is a term generally known in the art and refers to a ratio describing the amount of feed consumed per unit of production (i.e. gain, milk eggs). Enhancement of feed efficiency is an overall decrease in the ratio over that which would otherwise occur without implementation of the methods and/or administration of the compositions of the present invention.

Gain efficiency is a term generally known in the art and refers to a ratio of weight gain of an animal/weight of food ingested. Enhancement of gain efficiency is an overall increase in the ratio over that which would otherwise occur without implementation of the methods and/or administration of the compositions of the present invention.

Growth and enhancing growth are terms generally known in the art and refer to increases in either, or both, weight and size (e.g., height, width, diameter, circumference, etc.) over that which would otherwise occur without implementation of the methods and/or administration of the compositions of the present invention. Growth can refer to an increase in the mass (e.g., weight or size) of the entire animal or of a particular tissue (e.g., muscle tissue in general or a specific muscle). Alternatively, growth can indicate a relative increase in the mass of one tissue in relation to another, in particular, an increase in muscle tissue relative to other tissues (e.g., adipose tissue).

Reducing mortality refers to increasing the survivability or decreasing the death rate in animals after birth or hatch as compared with that which would otherwise occur in the absence of implementation of the methods and/or administration of the compositions of the present invention.

Enhanced breast or leg meat yield refers to increasing the amount of breast or leg meat in a poultry animal compared with that which would otherwise occur in the absence of implementation of the methods and/or administration of the compositions of the present invention.

Effective amount and effective rate refers to the amounts and rates of administration of enhanced lauric acid distillate, such as hydrolyzed lauric acid distillate, to provide enhanced growth, enhanced gain and/or feed efficiency, reduced mortality, and/or enhanced meat yield. Further, such amount and rates should result in no or few adverse events in the treated animal. As those familiar with the art will understand, the amounts and rates will vary depending upon a number of factors. These factors include, for example, the type of animal being treated, its weight and general physical condition, and the dosing regimen. Ranges for the rate of administration of enhanced lauric acid distillate, such as hydrolyzed lauric acid distillate, are from about 1 to about 3000, desirably 10 to 1000, and more desirably from about 10 to about 500, mg/kg of weight of the animal. These amounts are to be administered normally every day for at least 7 days, at least 2 weeks, at least 30 days, over 60 days, over 100 days, or for all or a substantial portion of the life of the animal.

Animal feed, as used herein, includes all solid or semi-solid feeds, as well as liquid feeds, and includes pre-mixes. The animal feed will be admixed with the distillate described above to form an animal feed composition which, when administered, will provide an effective amount of the distillate to the animal. Normally, the amount of the distillate will be from about 0.025 to about 2.5% by weight of the animal feed composition, desirably from about 0.1 to 2.0%, and more desirably from about 0.1 to 0.5%.

The compositions and methods of this invention may further include, in combination with the distillate, one or more other active ingredients. Other active ingredients include any material which can be added to the feed to enhance the animal's health, performance, and/or well-being. Examples of such include polyether ionophores feed additives such as monensin, salinomycin, narasin, lasalocid and laidlomycin; antibiotics such as the tetracyclines, bacitracin, avilamycin, nicarbazin, tylosin, tiamulin, lincomycin, virginiamycin, quinolone antibacterials and carbadox; melengesterol acetate; agents for the prevention or treatment of sub-acute rumen acidosis such as sodium bicarbonate, acarbose and other amylase or glucosidase inhibitors; carcass quality/anabolic agents such as ractopamine, salbutamol, almeterol, zilpaterol, and other beta, and selective beta, adrenergic ligands; enzymes, minerals, vitamins and other supplements. Other active ingredients also include anabolic agents such as zearanol, trenbolone acetate and oestradiol; growth hormones such as bovine somatotropin and porcine somatotropin; insecticides/endectocides such as ivermectin, spinosad, spinetoram, doramectin, moxidectin, abamectin and other macrocyclic lactones; anthelmintics such as monepantel, levamisole, albendazole and other benzimidazole carbamates, morantel, pyrantel; ectoparasiticides such as pyrethroids, arylpyrazoles, neonicotinoids. Another example of an additional active ingredient is Maxiban®, which contains narasin and nicarbazin. The person skilled in the art will recognize that the agents listed above are examples of a wide range of feed additives which may be used. Other examples are referred to in “2006 Feed Additive Compendium” and “Handbook of Feed Additives 2006”. Example formulations are provided as follows.

| Example Formulation 1 | (wt %) |
|---|---|
| CORN, FINELY GROUND | 20.000 |
| Wheat Midds By-Product 27-34% NDF | 15.000 |
| Soybean Meal-48% Protein | 17.050 |
| CALCIUM CARB | 9.467 |
| DISTILLERS GRAINS WITH SOLUBLES | 5.194 |
| Mono-dicalcium phosphate | 0.183 |
| Choline Chloride | 0.052 |
| Amino Acids | 0.156 |
| PrimaLac ® | 0.050 |
| SALT | 0.350 |
| Lignin-Based Binder | 0.500 |
| SOYBEAN OIL-SPRAYED | 1.600 |
| Citric Acid Anhydrous | 0.200 |
| Pigment | 0.026 |
| Corn By-product | 29.639 |
| Phytase | 0.027 |
| Yucca Extract (F) | 0.050 |
| Yeast | 0.025 |
| Marigold Extract (F) | 0.070 |
| PALOMYS ® (LAD) (F) | 0.150 |
| Broiler Vitamin Premix | 0.150 |
| Poultry Trace Mineral Specifications | 0.060 |
| | 100.000 |

| Example Formulation 2 Poultry Premix | (wt %) |
|---|---|
| DISTILLERS GRAINS WITH SOLUBLES | 74.8575 |
| Yucca Extract (F) | 2.0000 |
| Amino Acids | 4.9425 |
| Yeast | 1.0000 |
| Marigold Extract (F) | 2.8000 |
| PALOMYS ® (LAD) (F) | 6.0000 |
| Broiler Vitamin Premix | 6.0000 |
| Poultry Trace Mineral Specifications | 2.4000 |

| Example Formulation 3 | (wt %) |
|---|---|
| CORN, FINELY GROUND | 28.807 |
| Wheat Midds By-Product 27-34% NDF | 15.000 |
| Soybean Meal-48% Protein | 9.850 |
| CALCIUM CARB | 9.250 |
| DISTILLERS GRAINS WITH SOLUBLES | 3.738 |
| Phosphate-Mono Dicalcium | 0.250 |
| SOYBEAN OIL | 2.550 |
| CORN GLUTEN MEAL, 60% | 3.700 |
| Choline Chloride | 0.050 |
| Amino Acids | 0.0188 |
| PrimaLac ® | 0.050 |
| Wheat Red Dog By-Product 17-27% NDF | 15.000 |
| SALT | 0.336 |
| Binder | 0.500 |
| Citric Acid Anhydrous | 0.200 |
| Corn By-product | 10.000 |
| Phytase | 0.025 |
| Yucca Extract (F) | 0.050 |
| Yeast | 0.025 |
| Marigold Extract (F) | 0.070 |
| PALOMYS ® (LAD) (F) | 0.150 |
| Broiler Vitamin Premix | 0.150 |
| Poultry Trace Mineral Specifications | 0.060 |
| | 100.000 |

| Example Formulation 4 | (wt %) |
|---|---|
| CORN-FINE GROUND | 54.305 |
| Rice Bran | 10.000 |
| SOYBEAN MEAL 48% | 16.067 |
| CALCIUM CARB | 9.387 |
| DDGS | 2.284 |
| Phosphate-Mono Dicalcium | 0.480 |
| CORN GLUTEN MEAL | 3.400 |
| CHOLINE CHL-60 | 0.027 |
| PrimaLac ® | 0.050 |
| SALT | 0.340 |
| CITRIC ACID ANHYDROUS | 0.300 |
| Binder | 0.833 |
| PHYTASE | 0.027 |
| DISTILLERS GRAINS WITH SOLUBLES | 1.871 |
| Yucca Extract (F) | 0.050 |
| Amino Acids | 0.123 |
| Yeast | 0.025 |
| Marigold Extract (F) | 0.070 |
| PALOMYS ® (LAD)(F) | 0.150 |
| Broiler Vitamin Premix | 0.150 |
| Poultry Trace Mineral Specifications | 0.060 |
| | 100.000 |

### First Study

A first study in male broiler chickens (Ross 708) was undertaken to evaluate the effects of approximately 98% pure lauric acid (LA) and hydrolyzed palm kernel lauric acid (fatty acid) distillate (HPKFAD having approximately 50% lauric acid), as measured by growth performance when comparing non-zero dosages of LA and HPKFAD versus positive and negative controls. Growth performance measures were average daily gain (ADG) and feed intake (FI) as well as feed efficiency.

Male broiler chickens were obtained at less than 1 day of age, and were allotted to one of the following ten groups: 1) negative control; 2) positive control (salinomycin 50 g/T starter and grower, 0 g/T finisher/BMD 50 g/T starter, 25 g/T grower and finisher); 3) 0.25% HPKFAD; 4) 0.5% HPKFAD; 5) 1.0% HPKFAD; 6) 2.0% HPKFAD; 7) 0.1% LA; 8) 0.2% LA; 9) 0.4% LA; and 10) 0.8% LA. The percentage of HPKFAD or LA is the weight % of HPKFAD or LA in the feed. The common starter, grower, and finisher basal formulations, as well as the treatment diet replacement rates, were as follows:

Basal Diet Formulations

| | | Starter Base | Grower Base | Finisher Base |
|---|---|---|---|---|
| | | % | % | % |
| 14 | Corn AS 101 15 | 62.172 | 65.768 | 71.556 |
| 65 | Soybean Meal A | 30.710 | 27.003 | 21.093 |
| 120 | Pork Meat & Bo | 5.102 | 5.102 | 5.809 |
| 260 | Animal Fat 340 | 0.000 | 0.324 | 0.288 |
| 310 | Deflour Phosph | 0.498 | 0.356 | |
| 330 | Limestone | 0.460 | 0.418 | 0.320 |
| 340 | Salt | 0.422 | 0.440 | 0.477 |
| 355 | DL-Methionine | 0.245 | 0.237 | 0.179 |
| 365 | L-Lysine | 0.080 | 0.060 | 0.017 |
| 510 | Choline Chlori | 0.105 | 0.089 | 0.058 |
| 520 | Poultry Vitami | 0.102 | 0.102 | 0.102 |
| 620 | Poultry Trace | 0.102 | 0.102 | 0.102 |
| | | 100.00 | 100.00 | 100.00 |

Fat Replacement Rate for Treatment Diet

| | Trt. 1 | Trt. 2 | Trt. 3 | Trt. 4 | Trt. 5 | Trt. 6 | Trt. 7 | Trt. 8 | Trt. 9 | Trt. 10 |
|---|---|---|---|---|---|---|---|---|---|---|
| | % | % | % | % | % | % | % | % | % | % |
| Base Diet | 98-X | 98.00 | 98.00 | 98.00 | 98.00 | 98.00 | 98.00 | 98.00 | 98.00 | 98.00 |
| Fat | 2.00 | 2.00 | 1.75 | 1.50 | 1.00 | 0.00 | 1.90 | 1.80 | 1.60 | 1.20 |
| HPKFAD | | | 0.25 | 0.50 | 1.00 | 2.00 | | | | |
| LA | | | | | | | 0.10 | 0.20 | 0.40 | 0.80 |

The diet for the starter phase was provided on days 0-21; the diet for the grower phase was provided on days 21-35; and the diet for the finisher phase was provided on days 35-49; and comprised 17, 30, and 35% of the total feed, for the phases respectively. Measurements of the chickens were taken on days 0, 21, 35 and 49, while carcass evaluations were conducted on day 50.

Results to evaluate the effect of LA and HPKFAD at the specified doses used the following variables:

Average Daily Gain

Feed Efficiency

Average Daily Feed Intake (ADFI)

Average Breast Weight

Percent Breast Yield

Average Leg Weight

Percent Leg Yield

Average Hot Carcass Weight

In general, there were no positive treatment differences in overall ADG when comparing non-zero HPKFAD treatments versus negative control with the exception of HPKFAD included at 2%, in which case a decline in ADG was detected (P=0.050). LA, when included at 0.2 and 0.4%, indicated a decline in overall ADG (P 0.038).

HPKFAD improved overall feed efficiency when included at 0.5, 1.0 and 2.0% compared to negative control (P<0.001). LA improved overall feed efficiency when included at 0.4 and 0.8% (P>0.016) compared to negative control. In addition, both HPKFAD and LA had significant linear and plateau responses (P<0.001). Similar responses were detected in the starter and grower phases.

Both HPKFAD and LA showed a decrease in overall ADFI for all non-zero treatments versus negative control (P 0.045) with the exception of LA 0.1%, which was numerically lower but not statistically significant from negative control (P=0.166).

For both HPKFAD and LA, there were no treatment differences in Average Breast Weight for non-zero treatments versus control (P≧0.236). An increase was shown in Percent Breast Yield at the HPKFAD 0.5% dose level (P=0.087). No other treatment differences were observed for the other HPKFAD and LA treatments comparisons (P≧0.404).

For both HPKFAD and LA, there were no treatment differences in Average Leg Weight for non-zero treatments versus control (P≧0.267). An increase was shown in Percent Leg Yield at the HPKFAD 1% dose level (P=0.097). No other treatment differences were observed for the other HPKFAD and LA treatments comparisons (P≧0.195).

For both HPKFAD and LA, there were no differences (P≧0.111) for non-zero treatments versus control. In addition, there was a significant linear decrease response for HPKFAD (P=0.061).

Removed Animals (Adverse Event) was recorded. For both HPKFAD and LA, there were no differences in the overall proportion of animals removed (P≧0.170).

Overall, results of the study indicate HPKFAD improved overall feed efficiency when included at 0.5, 1.0, and 2% compared to negative control. LA improved overall feed efficiency when included at 0.4 and 0.8% compared to negative control. In addition, both HPKFAD and LA had significant linear and plateau responses. In general there were no positive treatment differences in overall Average Daily Gain (ADG), with the exception of 2% HPKFAD in which a decline in ADG was detected. Both HPKFAD and LA showed a decrease in overall Average Daily Feed Intake (ADFI) for all non-zero treatments versus negative control. HPKFD increased breast and leg yield at the 0.5 and 1% feeding rates, respectively, while LA did not so such an increase. For both HPKFD and LA, there were no differences in average hot carcass weigh for non-zero versus control. In summary, the HPKFAD was more effective than pure LA when compared on equivalent lauric acid inclusion levels.

### Second Study

A second study was conducted to evaluate the effects of four levels of lauric acid (as HPKFAD containing approximately 50% lauric acid) when fed to male Ross 308 broiler chickens, from day-old to slaughter at 42 days. The four doses were 0.25, 0.5, 1.0, and 2% HPKFAD in the complete feed, and measurements were taken at days 0, 12, 25, and 42. The birds were fed a standard commercial basal starter ration from 0-12 days, a grower ration from 12-25 days, and a finisher ration from 25-42 days. The feeds contained no growth promoters or anti-coccidial products. The study feed (short-cut pellets and pellets) were manufactured by Roslin Nutrition Ltd. Tables 2, 3, and 4 below display the basal diet composition, the calculated analysis, and the revised dietary soya oil inclusion, respectively.

TABLE 2
BASAL DIET COMPOSITION

| | Starter (kg) | Grower (kg) | Finisher (kg) |
|---|---|---|---|
| Wheat | 678.25 | 703.6 | 765.55 |
| Hipro soya | 250.0 | 225.0 | 185.0 |
| 66% Fish meal | 25.0 | 25.0 | 0.0 |
| Soya oil | 20.0 | 20.0 | 20.0 |
| Mono dical phos | 6.25 | 8.0 | 10.0 |
| Salt PDV | 2.5 | 2.5 | 3.0 |
| Lime flour | 10.0 | 8.0 | 6.25 |
| Lysine HCL | 1.5 | 1.5 | 3.2 |
| Methionine | 1.5 | 1.4 | 2.0 |
| Roslin str/gwr (min/vit)¹ | 5.0 | 5.0 | 5.0 |
| Total | 1000 | 1000 | 1000 |

¹Vitamin and mineral premix is added at 5 kg/tonne (0.5%) to provide the following nutrients per kg diet: 12,000 IU vitamin A; 5,000 IU vitamin D₃; 50 IU vitamin E; 3 mg vitamin K; 2 mg vitamin B₁; 7 mg vitamin B₂; 5 mg vitamin B₆; 15 mg vitamin B₁₂; 50 mg nicotinic acid; 15 mg pantothenic acid; 1 mg folic acid; 200 mg biotin; 80 mg iron; 10 mg copper; 100 mg manganese; 0.5 mg cobalt; 80 mg zinc; 1 mg iodine; 0.2 mg selenium; 0.5 mg molybdenum.

TABLE 3
Calculated Analysis

| | | |
|---|---|---|
| Dry matter | % | 87.208601 |
| A Oil | % | 3.619875 |
| B Oil | % | 4.334975 |
| Protein | % | 21.23725 |
| Fibre | % | 2.34215 |
| Ash | % | 5.521917 |
| DE | MJ/KG | 14.292775 |
| ME Poultry | MJ/KG | 12.3951 |
| LYS | % | 1.21854 |
| MET | % | 0.479835 |
| M + C | % | 0.8288 |
| THR | % | 0.76079 |
| TRY | % | 0.261172 |
| CALC | % | 0.962748 |
| TPHOS | % | 0.587508 |

TABLE 4
Revised Dietary Soya Oil Inclusion Levels (kg/1000 kg) to
Achieve Isocaloric Diets with HPKFAD

| | Neg | 0.25% | 0.5% | 1.0% | 2.0% |
|---|---|---|---|---|---|
| Diet | Control | HPKFAD | HPKFAD | HPKFAD | HPKFAD |
| Starter | 20.0 kg | 17.5 kg | 15.0 kg | 10.0 kg | 0.0 kg |
| Grower | 20.0 kg | 17.5 kg | 15.0 kg | 10.0 kg | 0.0 kg |
| Finisher | 20.0 kg | 17.5 kg | 15.0 kg | 10.0 kg | 0.0 kg |

Mortality for the negative control, 0.25%, 0.5%, 1.0%, and 2.0% treatments groups was 7.1%, 3.8%, 3.8%, 3.8%, and 4.4%, respectively. The commercial mortality average is 5%. The reductions in mortalities in the first three treatment groups were statistically significant, and differences were most marked during the 12-25 day period. Feed intakes were significantly reduced in the 0.25% and 1.0% treatments, with smaller but non-significant reductions in feed intake in the 0.5% and 2.0% treatment groups, as compared to negative control. There were no statistically significant effects on final liveweight, although 0.25% HPKFAD reduced liveweight by 43 g per bird, and 2.0% showed an increase in final liveweight of 55 g per bird. The highest dose, 2.0%, showed a significant increase in average weight gain compared to the negative controls (2408 g and 2310 g, respectively).

Administration of HPKFAD in the diets of broiler chickens improved feed efficiency at all the dose rates tested (0.033, 0.025, 0.063, and 0.054 for the 0.25%, 0.5%, 1.0%, and 2.0% treatments groups, respectively), but the differences were only statistically significant at the 1 and 2% dose rates.

### Third Study

A study in male Large White/Landrace cross pigs was conducted to evaluate the effects of four levels of lauric acid (as HPKFAD containing approximately 50% lauric acid) as compared to negative control when included in-feed on the liveweight gain and feed efficiency of commercial weaners over the grower/finisher phases for 84 day (approximately 30-100 kg live weight). The four doses were 0.25, 0.5, 1.0, and 2% HPKFAD. Measurements were taken at days 1, 29, 57, 83, and 85. Tables 5 and 6 are the basal diet composition and the revised dietary soya oil inclusion, respectively.

TABLE 5
BASAL DIET COMPOSITION

| | Grower (kg) | Finisher (kg) |
|---|---|---|
| Wheat 11%@ 76+kg/hl | 530.00 | 567.00 |
| Barley 66 kg/hl | 150.0 | 150.00 |
| Bulk Hipro Soya | 240.0 | 240.00 |
| Bag SA Fishmeal | 37.5 | — |
| Soya Bean Oil | 20.00 | 15.00 |
| M939 SCA Finisher + Lysine | 22.5 | 22.5 |
| Monocalcium phosphate | — | 5.00 |
| Salt PDV | — | 0.50 |
| Total | 1000 | 1000 |

TABLE 6
Revised Dietary Soya Oil Inclusion Levels (kg/1000 kg) to achieve
Isocaloric Diets with HPKFAD

| | | 0.025% LA | 0.05% LA | 0.1% LA | 0.2% LA |
|---|---|---|---|---|---|
| | | (0.5 kg | (1.0 kg | (2.0 kg | (4.0 kg |
| Diet | Neg Control | HPKFAD) | HPKFAD) | HPKFAD) | HPKFAD) |
| Grower | 20.0 kg | 19.5 kg | 19.0 kg | 18.0 kg | 16.0 kg |
| Finisher | 15.0 kg | 14.5 kg | 14.0 kg | 13.0 kg | 11.0 kg |

Results at day 83 are as follows in Tables 7 and 8:

TABLE 7
Average Daily Gain (kg)

| | | | | 83 day gain |
|---|---|---|---|---|
| Treatment | Block 1 | Block 2 | Overall | c.f. control (%) |
| Negative control | 0.958 | 0.922 | 0.940 | — |
| HPKFAD 0.5 kg/tonne | 0.985 | 0.933 | 0.959 | 1.58 (2.0) |
| HPKFAD 1.0 kg/tonne | 0.980 | 0.905 | 0.943 | 0.23 (0.3) |
| HPKFAD 2.0 kg/tonne | 0.983 | 0.916 | 0.949 | 0.77 (1.0) |
| HPKFAD 4.0 kg/tonne | 0.981 | 0.941 | 0.961 | 1.74 (2.2) |

TABLE 8
Feed Efficiency)

| | | | | 83 day feed |
|---|---|---|---|---|
| | | | | efficiency c.f. |
| Treatment | Block 1 | Block 2 | Overall | control (%) |
| Negative control | 2.644 | 2.325 | 2.484 | — |
| HPKFAD 0.5 kg/tonne | 2.547 | 2.326 | 2.436 | −0.048 (1.9) |
| HPKFAD 1.0 kg/tonne | 2.560 | 2.330 | 2.445 | −0.039 (1.6) |
| HPKFAD 2.0 kg/tonne | 2.553 | 2.360 | 2.457 | −0.027 (1.1) |
| HPKFAD 4.0 kg/tonne | 2.616 | 2.309 | 2.462 | −0.022 (0.9) |

### Fourth Study

A total of 96 pigs (initial BW of 9.98 lbs) were allotted to one of 8 treatments (12 replicates per treatment with 1 pigs per replicate). The treatments were 1) a negative control (NC) diet with no antibiotic (Ab), 2) a control diet with an Ab (Mecadox), 3) a diet with LAD at 0.25%, 4) a diet with LAD at 0.5%, 5) a diet with LAD at 1%, 6) a diet with calsporin, 7) a diet with calsporin and LAD at 1%, 8) and a diet with calsporin and LAD at 0.5%. Feed intakes and body weights were determined at the end of each feeding phase. A challenge model (a dirty room and aisle pigs) was used in this trial to see if this could elicit a negative response. Also, ZnO and CuSO₄ were not added into the diet to enhance the challenge. The feeding phases were from d 0 to 5, d 0 to 11, d 11 to 20, d 20 to 40. The data, especially from Phase 1 and 2, illustrated the pigs were severely challenged in this trial, which enhanced the effect of the antibiotic. Pigs fed the Ab had improved performance in this trial, whereas pigs fed calsporin had no effect. The optimum level of LAD was 0.25% for the early growth period and increased to 0.50% for the later growth period. Accordingly, the recommended level of HLAD to be fed to nursery pigs is between 0.25% and 0.5%. Adding LAD with calsporin had no additive effect on pigs in this trial.

### Fifth Study

A trial was conducted to evaluate the effect of hydrolyzed lauric acid distillate (HLAD) on growth performance of grow-finish pigs and to evaluate the effectiveness of atypical pen conditions for development of a challenge model. A total of 120 pigs (initial BW: 86 lbs and final BW: 258 lbs) were allotted to one of 6 dietary treatments (10 replicates consisting of five replicates of barrows and five replicates of gilts per treatment with two pigs per replicate). The treatment diets were a 1) control, 2) antibiotic bacitracin methylene disalicylate (BMD), 3) lauric acid (LA) at 0.1%, 4) HLAD at 0.1%, 5) HLAD at 0.2%, and 6) HLAD at 0.4%. Feed intakes and body weights were measured at the end of each of three feeding phase. A challenge of having two pigs per pen along with housing in a dirty room (the room was not cleaned after the previous study was concluded) was used in this trial to see if this could reduce the growth of pigs. The three feeding phases were approximately from 86 lbs to 127 lbs, 127 to 199 lbs, and 199 lbs to 258 lbs. Overall performance (ADG, ADFI, Feed efficiency) and BW at the end of any phase were not affected by diet. During Phase 1, pigs fed HLAD at 0.4% had an increased (P<0.05) Feed efficiency relative to those fed the antibiotic, HLAD at 0.1%, or HLAD at 0.2%. During Phase 2, pigs fed antibiotic had an increased (P<0.05) Feed efficiency relative to those fed the LA at 0.1% or the HLAD at 0.4%. Also, pigs fed the HLAD at 0.4% had a lower (P<0.05) Feed efficiency relative to those fed the HLAD at 0.1%. During Phase 3, pigs fed HLAD at 0.1% had an increased (P<0.05) ADG relative to those fed LA at 0.1%. Pigs fed LA at 0.1% had a decreased (P<0.05) ADFI relative to those fed the control diet or the diet with HLAD at 0.4%. The pigs in this trial did not show an effect to the challenge so deciding if and at what level HLAD can be fed to pigs for improved performance could not be determined.

### Sixth Study

This trial was conducted to test the effect of a distilled (DLAD) versus a hydrolyzed (HLAD) form of lauric acid in a necrotic enteritis challenge for broilers. Three different concentration of lauric acid was fed from 2 different sources. A positive control containing bacitracin methylene disalicylate (BMD) was used to establish growth potential and the various additives were added to a negative control diet with no antibiotic. All birds were challenged with a coccidian vaccine on day 7 and Clostridium Perfringens on days 14, 15, and 16. Body weight and feed intake were recorded on days 0, 13, and 22.

The birds fed BMD tended to have improved performance over birds fed the negative control. Throughout the study no improvement was noted with the addition of the hydrolyzed source of lauric acid.

During the challenge period birds fed increasing lauric acid from DLAD had a linear increase in body weight gain and a tendency for increase feed intake. The birds fed 0.143% and 0.200% DLAD had a tendency to have higher body weight compared to birds fed the negative control diets, while only birds fed the 0.143% DLAD had higher feed intake.

For the overall data, birds fed increasing levels of lauric acid had a linear increase in body weight gain. Birds fed the 0.143 and 0.200% DLAD tended to have higher body weight gain than birds fed the negative control diet. While birds fed 0.143% DLAD had a tendency for higher feed intake compared to birds fed the negative control. Birds fed the 0.200% DLAD tended to have improved feed efficiency compared to birds fed the negative diet and similar to birds fed the positive control diet with BMD.

The inconsistency of the different sources of lauric acid need to be further evaluated to obtain the benefits from this product.

Dietary Descriptions

| Treatments | Description |
|---|---|
| A | Negative Control |
| B | Positive Control-BMD |
| C | HLAD 0.1% |
| D | HLAD 0.175% |
| E | HLAD 0.25% |
| F | DLAD 0.082% |
| G | DLAD 0.143% |
| H | DLAD 0.2% |

### Seventh Study

This trial was conducted to determine the effect of hydrolyzed lauric acid (HLAD) using calcium soap (CHLAD) as a carrier on performance of broilers in challenge model. Also, the effect of protease alone and in combination with hydrolyzed lauric acid on performance of broilers in a challenge model. Birds were fed three levels of HLAD (0.10, 0.15, and 0.20%) and CHLAD (0.125, 0.1875, and 0.25%). Also, the midlevel of both sources of lauric acid was fed together and individually with papain. This trial was conducted as a typical necrotic enteritis trial with a coccidian challenge (10× active dose of vaccine) occurring on d 7 and Clostridium Perfringens added on day 12, 13, 14, and 15. Growth, feed intake, and mortality were recorded weekly.

ADG was decreased (P<0.05) in the diets that contained papain relative to those fed any other diet. Chicks fed the diet with 0.1% CHAD had an increased (P<0.05) ADG relative to those fed the negative control, 0.15% CHLAD, 0.20% CHLAD, 0.2% HLAD, and the combination of CHLAD and HLAD.

Feed intake was decreased (P<0.05) in the diets that contained papain relative to those fed any other diet. Chicks fed the diet with 0.1% CHAD had an increased (P<0.05) ADFI relative to those fed 0.15% CHLAD, 0.20% CHLAD and the combination of CHLAD and HLAD.

Feed efficiency was higher (P<0.05) in chicks fed the negative control (and challenged) relative to those fed all other diets except the diet that had papain and 0.15% HLAD (which had the highest feed efficiency).

The level of 0.15% HLAD tended to have a better ADG than the negative control which agrees with past data in our research facility. The overall best test treatment was with 0.1% CHLAD which tends to agree with the in vitro data which indicated that a lower level of CHLAD can have the same effect as HLAD.

| Treatments/ | | |
|---|---|---|
| Diets | Description | Challenged |
| A | Control | No |
| B | Negative Control | Yes |
| C | Positive Control-BMD | Yes |
| D | 0.1% HLAD | Yes |
| E | 0.15% HLAD | Yes |
| F | 0.2% HLAD | Yes |
| G | 0.125% CHLAD | Yes |
| H | 0.1875% CHLAD | Yes |
| I | 0.25% CHLAD | Yes |
| J | 0.1% HLAD + 0.125% CHLAD | Yes |

### Eighth Study

For this study broilers were placed in Petersime batteries and challenged under similar commercial farming conditions. These methods included a coccidian challenge in the feed on d 8 and then a 4 day challenge with Clostridia perfringens on d 12, 13, 14, and 15 to mimic conditions commonly found under commercial production. At the end of 21 days all broilers and feed were weighed to determine growth performance (gain, feed intake, feed efficiency). Also, the intestines were removed and weighed to determine if lauric acid changes the maintenance energy needed to maintain the intestine.

On day 7 chicks fed hydrolyzed lauric acid distillate at 0.2%+BMD (HLAD+BMD) had an increased (P<0.05) BW and gain relative to those fed any other dietary treatment. Chicks fed BMD had a higher (P<0.05) feed intake relative to those fed any other dietary treatment. Feed efficiency was lower (P<0.05) in chicks fed the 2% HLAD relative to those fed BMD. Chicks fed the 2% HLAD tended to have a lower feed efficiency relative to those fed the negative control (NC).

There was no effect of BW on day 13 or feed intake during this period in chicks fed any of the dietary treatments. Gain was decreased (P<0.05) in chicks fed the 2% HLAD relative to those fed the NC. Chicks fed the NC had a lower (P<0.05) feed efficiency relative to those fed the HLAD at 0.2%.

On day 21 chicks fed HLAD+BMD had an increased (P<0.05) BW and gain relative to those fed the NC. Also, chicks fed the 2% HLAD had an increased (P<0.05) BW gain relative to those fed the NC. There was no effect of feed intake during this period in chicks fed any of the dietary treatments. Chicks fed the 2% HLAD had a lower (P<0.05) feed efficiency relative to those fed the NC or those fed BMD. Also, chicks fed HLAD+BMD had a lower (P<0.05) feed efficiency relative to those fed the NC. Intestinal weights were not affected in this trial (trend to be lowest in the diet with BMD and LA with the majority coming from BMD).

There was no effect of treatment on overall feed intake or feed efficiency. Chicks fed HLAD+BMD had an increased (P<0.05) overall gain relative to those fed the NC.

There was not as good a challenge in this trial as in others noted by the fact that the BMD only tended to improve performance. Similar gain response was obtained with the birds fed 0.2% HLAD. The HLAD fed birds tended to have increased gain (especially in the 2% HLAD treatment) especially during the challenge period which has been seen in other trials here and in field research. The effect was similar if not slightly better than birds fed BMD.

During the first week, the addition of 0.2% HLAD with BMD showed an improved gain and feed intake. This effect was above and beyond either product fed alone. This was not expected because the challenge did not begin until day 14. Also, during the challenge period this same combination improved BW above the NC and above either additive alone. This resulted in an improved overall gain in chicks fed the combination of HLAD and BMD above those chicks fed the diets with either of these additives alone. This may mean that HLAD may have a different mode of action than just an antimicrobial agent. The reason to add a high level of HLAD in place of poultry fat was that lauric acid is a medium chain fatty acid (MCFA) and this fatty acid source may be able to improve energy utilization due to the fact that MCFA are preferentially moved into the mitochondria without further physiological break-down. Overall there was no negative impact on this substitution on growth performance (lower than NC in growth for day 7-13). However there was an improvement in feed efficiency during the periods of 0-7 d (tendency) and 13-21 d indicating that energy from feeding MCFA (especially for the first 7 d and during a challenge may be better utilized than feeding animal fat in chicks. There was no effect on intestinal weights in this trial with any of the dietary treatments which may have been due to the challenge being less than in past trials.

Overall this trial exhibited a limited response from a challenge perspective. HLAD fed at 0.2% or BMD tended to improve performance. However, the combination of the two showed an improved performance above feeding each alone. The addition of 2% HLAD did show some improvements in feed efficiency (not in the overall data but for specific periods of growth) indicating that this product may be better utilized from and energy perspective relative to poultry fat mostly likely due to a preferential move into the mitochondria.

### Ninth Study

The objective of this study is to determine the effect of 0.08 and 0.16% of distilled lauric acid distillate (DLAD) containing 70% lauric acid compared with a negative control when included in feed on the live weight gain and feed efficiency of commercial nursery pigs over the starter phase for 42 days (approximately 6-30 kg live weight). At days 0, 14, 28, and 42, the live weight is measured. All feed offered and weighed back is recorded.

Significant differences in average daily gain were recorded for DLAD at 0.08% during the first period (0-14 days) and at 0.16% during the second period (14-28 days). Overall there was a non-significant response of 6.2% and 6.7% to DLAD at 0.08% and 0.16% respectively.

While there were no significant improvements in feed efficiency in any period, there was a significant improvement overall for the 0.08% and 0.16% levels of 0.66% and 1.27% respectively.

Feed efficiency was seen to show a significant improvement overall for the 0.08% and 0.16% levels of 1.17% and 1.25% respectively, but not during any individual periods.

There were overall non-significant increases in daily feed intake of 4.97% and 5.36% for the 0.08% and 0.16% treatments respectively. The only significant difference appears in the 0.16% treatment between days 14 and 28.

All pigs were treated on Day 9 with Baytril® (i/m single injection) and Apralan Soluble in the drinking water for seven days as a treatment for salmonella.

### Tenth Study

The objective of this study was to determine the effect of three levels of lauric acid (0.025%, 0.05%, and 0.1%) compared with a negative control on the liveweight gain and feed efficiency of commercial swine over the grower/finisher phase and carcass measurements. Lauric acid was provided in feed as hydrolyzed palm kernel fatty acid distillate (HPKFAD or HLAD). Ractopamine was included in the ration (4.5 to 9.0 g/ton) for the last 45 to 90 lbs of gain for all pens (targeted end weight of approximately 290 lbs). Eight hundred thirty (830) barrows and gilts in 32 pens (25-27 animals/pen/8 pens/trt) were enrolled. Performance and carcass data are presented below. A significant improvement (P<0.05) was noted in average daily gain (ADG), average daily feed intake (ADFI), and hot carcass weight in animals consuming 0.025% lauric acid on a daily basis compared to the negative control group.

| | Lauric Acid Content (%) | | | | | |
|---|---|---|---|---|---|---|
| | 0 | 0.025 | 0.050 | 0.100 | SEM | P-Value |
| ADG, lb | 2.21^{a} | 2.29^{b} | 2.22^{ab} | 2.22^{ab} | 0.026 | 0.049 |
| ADFI, lb | 5.40^{a} | 5.60^{b} | 5.39^{ab} | 5.49^{ab} | 0.056 | 0.028 |
| Feed | 2.44 | 2.45 | 2.43 | 2.48 | 0.029 | 0.313 |
| Efficiency | | | | | | |
| Hot Carcass | 211.86^{a} | 218.95^{b} | 213.74^{ab} | 213.48^{ab} | 1.762 | 0.043 |
| Weight, lb | | | | | | |
| Back fat, in | 0.70 | 0.69 | 0.67 | 0.70 | 0.018 | 0.55 |
| Loin Depth, | 2.53 | 2.55 | 2.59 | 2.54 | 0.041 | 0.75 |
| in | | | | | | |
| Lean, % | 55.8 | 55.8 | 55.6 | 55.8 | 0.407 | 0.98 |

^{a,b}Means with different superscript differ (P < 0.05). Comparisons between non-zero doses were not tested.
*Note:
Lauric acid constitutes approximately 50% of the formulation HPKFAD.

### Eleventh Study

One hundred and ninety two (192) pigs were included in a randomized block study to determine the effect on growth, feed efficiency and carcass composition of two concentrations of Distilled Lauric Acid Distillate (DLAD) when compared to a negative control group. DLAD was included in the feed at 0, 0.4 and 0.8% during both the grower and the finisher phases. The study was conducted in a grower-finisher unit comprising 24 pens with 8 replicates per treatment.

During the grower or the finisher phase and over the whole study period, the pigs fed DLAD did not show significantly improved average daily weight gain ADG), feed intake (ADFI), feed efficiency (FE) or carcass composition when compared to the negative control pigs.

The number of pigs with health events or the number of Serious Adverse Events was also similar between treatment groups.

| | | | | Difference |
|---|---|---|---|---|
| | | | | from |
| | Control | 0.4‰ DLAD | 0.8‰ DLAD | control (*) |
| ADG (kg) | 1.003 | 0.987 | 0.999 | N.S. |
| ADFI (kg) | 2.679 | 2.678 | 2.717 | N.S. |
| Feed Efficiency | 2.676 | 2.716 | 2.722 | N.S. |
| Carcass weight (kg) | 92.958 | 92.178 | 92.278 | N.S. |
| Back fat (cm) | 14.817 | 15.554 | 14.667 | N.S. |
| Muscle depth (cm) | 57.939 | 59.088 | 58.850 | N.S. |

(*) N.S.: not significant

We claim:

1. A method for enhancing feed efficiency, enhancing gain, or reducing mortality, in an animal in need thereof comprising providing to said animal for an effective time an effective amount of enhanced lauric acid distillate, and optionally one or more other active ingredients.

2. The method of claim 1 wherein said effective amount of the distillate is between 1 and 3000 mg/kg of weight of said animal.

3. The method of claim 2 wherein said effective time is daily for at least 7 consecutive days

4. The method of claim 3 wherein said animal is a food animal and is a turkey, chicken, or pig.

5. The method of claim 1 wherein said providing is carried out using an animal feed having said distillate therein and optionally other active ingredients.

6. The method of claim 5 wherein the amount of distillate in the animal feed is between about 0.025 to about 2.5% by weight of the animal feed.

7. An animal feed composition for enhancing feed efficiency, enhancing gain, or reducing mortality in an animal in need thereof comprising animal feed and an effective amount of enhanced lauric acid distillate, and optionally one or more other active ingredients.

8. The animal feed composition of claim 7 wherein the amount of distillate in the animal feed is between about 0.025 to about 2.5% by weight of the animal feed.

9. The animal feed composition of claim 7 wherein the effective amount of distillate is such that said animal receives the distillate in an amount of between 1 and 3000 mg/kg of weight of said food animal when fed said animal feed composition.

10. The animal feed composition of claim 9 wherein said animal feed composition is adapted to provide an effective amount of distillate daily.

11. The animal feed composition of claim 10 wherein said animal feed is adapted to be fed to said animal for at least 7 consecutive days.

12. The animal feed composition of claim 7 wherein it is for a food animal and said food animal is a chicken, turkey, pig, fish, or shrimp.

13. A method for enhancing breast or leg meat yield in a poultry food animal in need thereof comprising providing to said poultry food animal for an effective time an effective amount of enhanced lauric acid distillate, and optionally one or more other active ingredients.

14. The method of claim 13 wherein said effective amount of the distillate is between 1 and 3000 mg/kg of weight of said poultry food animal.

15. The method of claim 14 wherein said effective time is daily for at least 7 consecutive days

16. The method of claim 14 wherein said poultry food animal is a turkey or chicken.

17. The method of claim 13 wherein said providing is carried out using a poultry animal feed having said distillate therein and optionally other active ingredients.

18. The method of claim 17 wherein the amount of distillate in the poultry animal feed is between about 0.025 to about 2.5% by weight of the animal feed.

19. A poultry animal feed composition for enhancing breast or leg meat yield in a poultry food animal in need thereof comprising poultry animal feed and an effective amount of enhanced lauric acid distillate, and optionally one or more other active ingredients.

20. The poultry animal feed composition of claim 19 wherein the amount of distillate in the animal feed is between about 0.025 to about 2.5% by weight of the animal feed.

21. The poultry animal feed composition of claim 19 wherein the effective amount of distillate is such that said poultry food animal receives the distillate in an amount of between 1 and 3000 mg/kg of weight of said poultry food animal when fed said poultry animal feed composition.

22. The poultry animal feed composition of claim 21 wherein said poultry animal feed composition is adapted to provide an effective amount of distillate daily.

23. The poultry animal feed composition of claim 22 wherein said animal feed is adapted to be fed to said food animal for at least 7 consecutive days.

24. The poultry animal feed composition of claim 23 wherein it is for chickens or turkeys.

25. A lauric acid distillate composition which has been enhanced by increasing the overall lauric acid content to up to 75%, by being hydrolyzed, and/or by being further distilled, and optionally one or more other active ingredients.

26. The enhanced lauric acid distillate composition of claim 25 wherein it is hydrolyzed lauric acid distillate.

27. The hydrolyzed lauric acid distillate composition of claim 26 wherein the conversion of the hydrolysis is at least 40%.

28. The hydrolyzed lauric acid distillate composition of claim 27 wherein the conversion of the hydrolysis is substantially 100%.
